SEQUENCE LISTING (22 sequences)

SEQ ID NO: 1 - protein, 9 aa, Homo sapiens
  DOMAIN: TRAF6 binding domain from human CD40
KQEPQEINF

SEQ ID NO: 2 - protein, 9 aa, Unknown
  DOMAIN: TRAF6 binding domain from mouse CD40
RQDPQEMED

SEQ ID NO: 3 - protein, 9 aa, Homo sapiens
  DOMAIN: TRAF6 binding domain from human RANK
RQMPTEDEY

SEQ ID NO: 4 - protein, 9 aa, Homo sapiens
  DOMAIN: TRAF6 binding domain from human RANK
FSEPLEVGE

SEQ ID NO: 5 - protein, 9 aa, Homo sapiens
  DOMAIN: TRAF6 binding domain from human RANK
RNPPGEDCE

SEQ ID NO: 6 - protein, 9 aa, Unknown
  DOMAIN: TRAF6 binding domain from mouse RANK
RKIPTEDEY

SEQ ID NO: 7 - protein, 9 aa, Unknown
  DOMAIN: TRAF6 binding domain from mouse RANK
FQEPLEVGE

SEQ ID NO: 8 - protein, 9 aa, Unknown
  DOMAIN: TRAF6 binding domain from mouse RANK
GNTPGEDHE

SEQ ID NO: 9 - protein, 9 aa, Homo sapiens
  DOMAIN: TRAF6 binding domain from human IRAK-1
PPSPQENSY

SEQ ID NO: 10 - protein, 9 aa, Homo sapiens
  DOMAIN: TRAF6 binding domain from human IRAK-1
PNQPVESDE

SEQ ID NO: 11 - protein, 9 aa, Homo sapiens
  DOMAIN: TRAF6 binding domain from human IRAK-1
RQGPEESDE

SEQ ID NO: 12 - protein, 9 aa, Unknown
  DOMAIN: TRAF6 binding domain from mouse IRAK
SPSPQENSY

SEQ ID NO: 13 - protein, 9 aa, Unknown
  DOMAIN: TRAF6 binding domain from mouse IRAK
PNQPVESDE

SEQ ID NO: 14 - protein, 9 aa, Unknown
  DOMAIN: TRAF6 binding domain from mouse IRAK
SQGPEESDE

SEQ ID NO: 15 - protein, 9 aa, Homo sapiens
  DOMAIN: TRAF6 binding domain from human IRAK-2
SNTPEETDD

SEQ ID NO: 16 - protein, 9 aa, Homo sapiens
  DOMAIN: TRAF6 binding domain from human IRAK-2
PLLPTENGE

SEQ ID NO: 17 - protein, 9 aa, Homo sapiens
  DOMAIN: TRAF6 binding domain from human IRAK-M
PSIPVEDDE

SEQ ID NO: 18 - protein, 9 aa, Homo sapiens
  DOMAIN: TRAF6 binding domain from human RIP2
IYMPPENYE

SEQ ID NO: 19 - protein, 34 aa, Unknown
  PEPTIDE: Peptide L-T6Bp-1
AAVALLPAVLLALLAPRKIPTEDEYTDRPSQPST

SEQ ID NO: 20 - protein, 30 aa, Unknown
  PEPTIDE: Peptide L-T6Bp-2
AAVALLPAVLLALLAPIPPFQEPLEVGEND

SEQ ID NO: 21 - protein, 19 aa, Unknown
  PEPTIDE: Peptide T6Bp-1
CRKIPTEDEYTDRPSQPST

SEQ ID NO: 22 - protein, 15 aa, Unknown
  PEPTIDE: Peptide T6Bp-2
CIPPFQEPLEVGEND

(19) United States
(12) Patent Application Publication
(10) Pub. No.: US 2004/0167072 A1
(43) Pub. Date: Aug. 26, 2004

(54) Inhibitors of receptor activator of NF-kappaB and uses thereof

(76) Inventors: Bharat B. Aggarwal, Houston, TX (US); Bryant G. Darnay, Houston, TX (US); Sujay Singh, San Diego, CA (US)

Correspondence Address: Benjamin Aaron Adler, ADLER & ASSOCIATES, 8011 Candle Lane, Houston, TX, 77071, US

(21) Appl. No.: 10/786,316

(22) Filed: Feb. 25, 2004

Related U.S. Application Data
(63) Continuation-in-part of application No. 10/143,293, filed on May 10, 2002.
(60) Provisional application No. 60/290,429, filed on May 11, 2001.

(51) Int. Cl. A61K 38/16; C07K 14/16

(52) U.S. Cl. 514/12; 530/350

(57) ABSTRACT

The present invention provides a RANK (receptor activator of NF-κB) inhibitor consisted of a TRAF-6 (TNF receptor-associated factor-6) binding domain attached to a leader sequence. The decoy peptide inhibits RANKL (RANK ligand)-mediated osteoclast differentiation, thus indicating that targeted disruption of interaction between RANK and TRAF6 may prove useful as a therapeutic for metabolic bone disorders, leukemia, arthritis, and metastatic cancer of the bone.

### CROSS-REFERENCE TO RELATED APPLICATION

This is a continuation-in-part application of patent application 10/143,293, filed May 10, 2002, which claims benefit of priority of provisional patent application 60/290,429, filed May 11, 2001, now abandoned.

### BACKGROUND OF THE INVENTION

1. Field of the Invention

The present invention relates generally to the fields of cytokine biology and bone diseases. More specifically, the present invention provides inhibitors of receptor activator of NF-κB (RANK) for therapy of disorders such as diseases associated with bone resorption.

2. DESCRIPTION OF THE RELATED ART

Members of the TNF and TNF receptor superfamilies play critical roles in the initiation and regulation of the immune response. Although members of these families share many overlapping biological functions, it appears from gene knockout studies that they have unique features.

One such receptor/ligand pair, receptor activator of NF-κB (RANK) and its ligand (RANKL/TRANCE/ODF/OPGL), is critically involved in regulation of bone remodeling and osteoclastogenesis. From knockout gene studies in mice, RANKL also functions in lymph node organogenesis and lymphocyte development. Furthermore, receptor activator of NF-κB and RANKL are implicated in the interactions between T cells and dendritic cells during the immune response.

As indicated by its name, receptor activator of NF-κB stimulates activation of nuclear factor-KB (NF-κB), a transcription factor that regulates the expression of a large number of genes that play essential roles in immune and inflammatory responses. Evidence over the past several years has indicated that some of the TNF receptor family members interact with a family of adapter proteins known as TRAFs, (TNF receptor-associated factors), which participate in activation of the transcription factor NF-κB and c-Jun N-terminal kinase (JNK).

The TNF receptor-associated factor family consists of six distinct proteins, each of which possesses a C-terminal homologous domain that is critical for self-association and is required for interaction with the receptors. All of the TNF receptor-associated factors, except for TRAF1 and TRAF4, also contain ring and zinc finger motifs in their N-termini, which appear to be utilized for interacting with other signaling molecules. Similar to the trimeric structure of the ligands and receptors of the TNF family, the C-terminus of the TRAF2 adapts a trimeric structure, as reported for TRAF2 in its interaction with peptides derived from TNF receptor 1 and CD40. This trimeric structure of the TNF receptor-associated factor molecules likely enables them to associate with downstream adapter proteins.

It has been demonstrated that TRAF2, TRAF5, and TRAF6 interact with receptor activator of NF-κB (RANK) and that receptor activator of NF-κB could activate both the NF-κB and JNK pathways. Subsequently, a more detailed analysis of the interaction of these TNF receptor-associated factors with receptor activator of NF-κB was reported.

A novel TRAF6-binding motif has been identified in RANK that is distinct from the TRAF2- and TRAF5- binding domains. A homologous TRAF6-binding motif in CD40 was described using a combinatorial peptide library approach (Pullen et al., 1999). The TRAF6 binding domain in RANK was sufficient for activation of NF-κB, suggesting that TRAF2 and TRAF5 are not necessary for NF-κB activation. However, it appears that the TRAF2-binding motif is sufficient for JNK activation, although the TRAF6-binding domain could also activate JNK, albeit to a lesser extent. Additionally, NIK (NF-κB inducing kinase) was also found to be required for the activation of NF-κB by receptor activator of NF-κB. In addition to TRAF2, TRAF5, and TRAF6, it has been demonstrated that TRAF1 and TRAF3 also associate with the carboxy terminus of receptor activator of NF-κB.

The role played by each TRAF molecule in RANK signal transduction remains elusive. Dominant negative mutants of TRAF2, TRAF5, and TRAF6 have been used to evaluate their role in NF-κB activation by RANK. It appears that all of the dominant negative TRAFs differentially inhibit the activation of NF-κB induced by overexpression of RANK in 293 cells. However, inclusion of all dominant negative mutants of TRAF2, TRAF5, and TRAF6 did not completely eliminate the activation of NF-κB induced by RANK in 293 cells. Stimulation of RANK also caused the recruitment of TRAF6, which in turn recruits and activates c-Src, which appears to be responsible for activation of phosphoinositol-3-kinase and protein kinase B/AKT, a molecule potentially involved in cell survival.

Knockout mouse models of RANKL, RANK, and osteoprotegerin have demonstrated an essential role of these molecules in osteoclastogenesis. The biological importance of these molecules is underscored by the induction of severe osteoporosis by targeted disruption of osteoprotegerin and by the induction of osteopetrosis by targeted disruption of RANKL or by overexpression of osteoprotegerin. Thus, osteoclast formation may be attributed to the relative ratio of RANKL to osteoprotegerin in the microenvironment of bone marrow, and alterations in this balance may be a major cause of bone loss in many metabolic bone disorders.

Similar to RANKL−/− mice, targeted disruption of receptor activator of NF-κB also lead to an osteopetrotic phenotype. Both RANK−/− and RANKL−/− mice exhibited absence of osteoclasts, indicating the essential requirement of these molecules for osteoclastogenesis. Additionally, mice lacking TRAF6, c-Src, c-Fos, or the NF-κB subunits p50/p52 also display an osteopetrotic phenotype. Although these mutant mice have osteoclasts, these cells apparently have defects in bone resorption. Thus, RANKL and receptor activator of NF-κB as well as their cytoplasmic signaling molecules are required for osteoclastogenesis.

Of the TRAF molecules that bind to receptor activator of NF-κB, only TRAF6 appears to be essential for osteoclast differentiation as indicated in mice lacking TRAF6. Thus, the interaction of receptor activator of NF-κB with TRAF6 may be a unique target for therapeutic intervention, and the ability to disrupt this interaction by a competitive, cell permeable peptide remains to be investigated.

The prior art is deficient in a method of disrupting the interaction between receptor activator of NF-κB and TRAF6 in order to inhibit RANKL signaling and osteoclast differentiation induced by RANKL. Such inhibitor would be useful as therapeutic in bone disorders and cancer associated with increased bone resorption. The present invention fulfills this long-standing need and desire in the art.

### SUMMARY OF THE INVENTION

TRAF6 (TNF receptor-associated factor 6) is a critical adapter protein for Receptor Activator of NF-κB (RANK) signaling. The present invention develops a novel TRAF6 decoy peptide (T6DP) with and without a peptide leader sequence that allows for transversing cellular membrane. Results shown below indicate that TRAF6 decoy peptide inhibits early events associated with RANKL signaling and RANKL-mediated osteoclast differentiation only when the leader sequence is attached. These data indicate that targeted disruption of interaction between RANK and TRAF6 may prove useful as a therapeutic for metabolic bone disorders, leukemia, arthritis, and metastatic cancer of the bone.

The present invention provides polypeptides that inhibit signaling mediated by TNF receptor-associated factor 6 (TRAF6), wherein said polypeptides comprise of a TRAF6 binding domain and a leader signal sequence. The present invention is further drawn to methods of inhibiting Receptor Activator of NF-κB Ligand (RANKL)-induced osteoclast differentiation using the polypeptides disclosed herein.

In another aspect of the present invention, there is provided a non-peptide analog that mimics the function of the decoy polypeptide disclosed herein, wherein said non-peptide analog inhibits signaling mediated by TRAF6. The present invention is further drawn to methods of inhibiting Receptor Activator of NF-κB Ligand (RANKL)-induced osteoclast differentiation using a non-peptide analog that mimics the function of the decoy polypeptide disclosed herein.

In another aspect of the present invention, there is provided a method of identifying a non-peptide small molecule capable of inhibiting interaction between receptor activator of NF-κB (RANK) and TNF receptor-associated factor 6 (TRAF6), comprising the step of: preparing a polypeptide comprising a TRAF6 binding domain; and examining binding of TRAF6 to said polypeptide in the presence and absence of a non-peptide small molecule, wherein reduced binding in the presence of said non-peptide small molecule would indicate that said non-peptide small molecule is capable of inhibiting RANK-TRAF6 interaction.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

FIGS. 1A-B show the novel TRAF6 binding domain in RANK. FIG. 1A shows the consensus sequence of the TRAF6 binding domain. The region within the amino acid sequence of CD40 (residues 230-245) that specifically interacts with TRAF6 (38) was used as the template for alignment of RANK, IRAK1, and IRAK2. Identical residues are in bold. A consensus motif is shown on the bottom.

FIG. 1B shows the peptides used in the present study. The hydrophobic domain of Kaposi fibroblast growth factor signal sequence (underlined) (36) was attached to the two potential TRAF6 binding domains from murine receptor activator of NF-κB. Additionally, two peptides lacking the leader sequence were synthesized.

FIGS. 2A-C show L-T6DP-1 inhibits RANKL-mediated osteoclast differentiation in RAW264.7 cells. FIG. 2A shows RANKL-induces TRAP positive osteoclasts. RAW264.7 cells were plated in 12-well plates and stimulated with RANKL (30 ng/ml) for 4 days. Cells were stained for tartrate resistance acid phosphatase essentially as described below. Photographs were taken using a 10×objective lens.

FIG. 2B shows L-T6DP-1 inhibits osteoclast differentiation. RAW cells were plated as described in FIG. 2A and treated with RANKL in the presence of 1, 30, or 100 μM of peptides as indicated. On day 4 cells were stained and evaluated as in FIG. 2A.

FIG. 2C shows L-T6DP-1 inhibits the total number of tartrate resistance acid phosphatase positive osteoclasts induced by RANKL. RAW264.7 cells were plated in 48-well plates in triplicate and treated with RANKL in the presence of increasing amounts of the indicated peptides. After 4-5 days, the cells were stained for tartrate resistance acid phosphatase and the total numbers of osteoclasts were counted as described below.

FIGS. 3A-B show leader TRAF6-binding peptide inhibits normal mouse derived osteoclast differentiation by RANKL and M-CSF. FIG. 3A shows mouse-derived monocytes were plated in 48-well plates in triplicate and costimulated with murine M-CSF (10 ng/ml) and RANKL (30 ng/ml) in the absence (0) or presence of the indicated peptides. After 6-7 days, the cells were fixed and stained for TRAP. The numbers of multi-nucleated, tartrate resistance acid phosphatase positive osteoclasts were counted.

FIG. 3B shows L-T6DP-1 inhibits osteoclast differentiation of mouse-derived monocytes. Mouse-derived monocytes were plated in 48-well plates and costimulated as in FIG. 3A with murine M-CSF (10 ng/ml) and RANKL (30 ng/ml) in the absence (left panel) or presence of the indicated peptides. After 6-7 days, the cells were fixed and stained for tartrate resistance acid phosphatase. A representative field from each well was taken at a magnification of 10×.

FIGS. 4A-C show that L-T6DP-1 specifically inhibits RANKL-induced NF-κB activation and TRAF6 binding. FIG. 4A shows inhibition of RANKL-mediated NF-κB activation by L-T6DP-1. RAW264.7 cells were plated in 6-well plates and treated with the indicated peptides for 5 h, then treated with RANKL (10 nM) for 15 min. Nuclear and cytoplasmic extracts were prepared and a gel mobility shift assays was performed with 8 μg of nuclear extracts as described below.

FIG. 4B shows inhibition of IκB degradation by L-T6DP-1. Cytoplasmic extracts (30 μg) from FIG. 4A were immunoblotted with anti-IκB as described below.

FIG. 4C shows L-T6DP-1 inhibits TRAF6 from binding to the receptor activator of NF-κB cytoplasmic domain. Cellular extracts from human 293 cells transfected with either FLAG-TRAF2, -TRAF5, or -TRAF6 were mixed with GST or GST-RANKcd in the absence or presence of 100 μM of the indicated peptides. A GST-pull down assay was performed as described below. The bound TRAFs were visualized by immunoblotting with anti-FLAG antibodies.

FIGS. 5A-C show L-T6DP-1 specifically inhibits RANKL-induced JNK, ERK, and p38 kinase activation. Figure SA shows inhibition of RANKL-mediated JNK activation by L-T6DP-1. RAW264.7 cells were plated in 6-well plates and treated with the indicated peptides for 6 h, then treated with RANKL (10 nM) for 15 min. Whole cell extracts were prepared and 30 μg of cell lysate was immunoprecipitated with anti-JNK1. In vitro kinase assays were performed using GST-Jun (1-79) as the substrate as described below.

FIG. 5B and FIG. 5C show inhibition of RANKL-mediated ERK and p38 activation by L-T6DP-1. Whole cell extracts (30 μAg) from FIG. 5A were separated by 10% SDS-PAGE and electroblotted onto a nitrocellulose membrane. The membranes were first immunoblotted with the indicated phospho-specific antibody and then stripped and re-probed with the indicated antibodies as described below.

FIGS. 6A-C show L-T6DP-1 inhibits osteoclast differentiation induced by breast cancer cells. FIG. 6A. Breast cancer cells (500/well) were plated in 24-well plates in the absence or presence of RAW cells (10000/well) and in the absence or presence of L-T6DP-1 or T6DP-1 (100 μM). After 5 days, the cells were fixed, stained for tartrate resistance acid phosphatase, and photographed with a 10x objective lens.

FIG. 6B shows RAW cells cultured alone or in the presence of the indicated cells on synthetic bone slides (BD BiCoat Osteologic MultiTest Slides). After 6 days, the cells were removed by washing the slide in bleach for 5 min and then washing thoroughly with distilled water. The slide was air dried and then photographs were taken with a light microscope (10× objective lens). The ghosts of osteoclasts (arrow) can be seen where functional osteoclasts destroyed the synthetic bone matrix.

FIG. 6C shows normal breast epithelial cells (MCF10A, 1000/well) grown in the presence of RAW and stained for tartrate resistance acid phosphatase as indicted in FIG. 6A. The right panel includes the addition of RANKL (100 ng/ml) to the co-culture.

FIGS. 7A-F show the structure of TRAF6. FIG. 7A. Ribbon diagram of the TRAF domain of TRAF6 in complex with TRANCE-R, shown as a trimeric model generated from the monomeric TRAF6. structure by applying the putative three-fold symmetry as observed in TRAF2 structures. Yellow: helices; Magenta: loops; Cyan, green and purple: β-strands from the three protomers respectively. Stick model: TRANCE-R peptide.

FIG. 7B shows worm Cα traces of superimposed TRAF6 (cyan) in complex with the TRANCE-R peptide (red), Apo-TRAF6 (yellow) and TRAF2 (blue) in complex with the core CD40 peptide (green), shown in a similar orientation as the cyan protomer in FIG. 7A. The distinct peptide directions in the two complexes are apparent.

FIG. 7C shows surface representation of TRAF6, colored based on electrostatic potential (−10 k_{b}T/e to 10 k_{b}T/e, where k_{b}, T and e are the Boltzmann constant, temperature and the electron charge respectively). Negatively and positively charged regions are shown in red and blue, respectively. The bound TRANCE-R peptide is shown as a stick model, with gray for carbon, cyan for nitrogen, magenta for oxygen and green for sulfur. This orientation is obtained by rotating 900 around the horizontal axis in FIG. 7B.

FIG. 7D shows surface representation of TRAF2, shown with the bound core CD40 peptide. Same orientation as in FIG. 7C.

FIG. 7E shows the interaction between TRAF6 (carbon atoms in white) and TRANCE-R peptide (carbon atoms in yellow), both shown as stick models. Main chain hydrogen bonds between the TRANCE-R peptide and the β7 strand of TRAF6 are shown as dotted lines. Residues in TRAF6 are labeled in red and those in TRANCE-R in blue. Side chains of some of the β7 residues are omitted for clarity.

FIG. 7F shows superposition of the TRANCE-R peptide (carbon atoms in yellow) with the CD40 peptide (carbon atoms in white). The P₃ residue of CD40 may have been influenced by crystal packing.

FIG. 8 shows sequence alignment of the TRAF-C domain of TRAF6 and TRAF2. Residues of TRAF6 and TRAF2 involved in receptor interactions are colored. Red: residues with less than 10% side chain surface exposure and more than 20Å² buried interfacial surface area; magenta: residues with 10-40% side chain surface exposure and more than 20Å² buried interfacial surface area; green: remaining interface residues. TRAF6 residues that completely abolished CD40 binding when mutated to alanines are labeled by filled circles, while those that did not are shown by open circles.

FIGS. 9A-C show osteolytic lesions induced with a breast cancer cell line in the tibia of a mouse. FIG. 9A. Radiographic image of a nude mouse tibia injected with human breast cancer cells (SUM149) forming an osteolytic tumor (arrow). FIGS. 9B and C show sections of the tumor in the tibia stained for TRAP⁺ cells (red), which are located at the interface between the tumor and bone at 100× (FIG. 9B) or 200× (FIG. 9C) magnification.

FIG. 10 shows L-T6DP-wt prevents breast cancer-induced ostelotyic lesions. Nude mice were injected with SUM149it1 breast cancer cells into the tibia with a dose of cells that had previously resulted in lytic tumors in 100% of animals. Beginning three days after injection, the mice received an i.p. injection 3 times per week of either 0.1 ml PBS (upper panels), or 1 mg of the L-T6DP-wt peptide in 0.1 ml of PBS (lower panels). The mice were weighed weekly, and X-ray images of the tibias were taken on day 28 after injection and at 2-week intervals thereafter. The mice received 29 injections over a period of 10 weeks. The X-ray images shown were taken after the 10 week period. Results of histology analysis are indicated below each mouse.

FIG. 11 shows mutations at P-2, P0 and P3 positions of a TRAF6-binding site in full-length TRANCE-R produced no effects on NF-kB activation.

FIG. 12 shows the effect of triple alanine mutation of L-T6DP-1. RAW264.7 cells were treated with various doses of peptides for 5 days. Cell viability was determined by crystal violet assays. L-T6DP-1-WT is the wild type peptide. T6DP-1 is the same peptide without the leader sequence and therefore does not enter cells. L-T6DP-1-Mut is the triple alanine mutant of L-T6DP-1-WT at positions P-2, P0 and P3 of the TRAF6-binding motif.

### DETAILED DESCRIPTION OF THE INVENTION

Receptor activator of NF-κB (RANK), a recently described member of the TNF receptor superfamily, is expressed primarily by dendritic cells, osteoclast progenitors, activated B and T cells and osteoclast. By binding to its ligand (RANKL), RANK causes the sequential recruitment of adapter molecules responsible for activation of signaling processes. These pathways lead to activation of protein kinases, which in turn activate transcription factors leading to changes in gene expression that alter the function of the cell.

Knockout mouse models of RANKL, RANK, and osteoprotegerin (OPG), a secreted soluble receptor that binds RANKL, have demonstrated an essential role of these molecules in osteoclastogenesis (i.e., bone remodeling). The biological importance of these molecules is underscored by the induction of severe osteoporosis by targeted disruption of osteoprotegerin and by the induction of osteoperosis by targeted disruption of RANKL, RANK, or by transgenic expression of osteoprotegerin. These results indicate that osteoclast formation may be attributed to the relative ratio of RANKL to osteoprotegerin in the microenvironment of bone marrow, and alterations in this balance may be a major cause of bone loss in many metabolic bone disorders. Hence, RANK/RANKL/osteoprotegerin have a major role in bone diseases and cancer-induced bone destruction that are due to increased osteoclastic activity.

In addition to osteoporosis, recent reports suggest a potential role of these molecules in other diseases including rheumatoid arthritis, giant cell tumor of bone, Paget's disease, and familial expansile osteolysis (due to a mutation in exon 1 of RANK). A T cell lymphoproliferative disorder has also been identified in which dysregulation of RANK and RANKL contributes to the survival of malignant T cell clone.

It has been recognized that breast and prostate cancers have the ability to invade and grow as metastases in bone causing osteolytic lesions. In metastatic tumor mouse models in which the tumor causes increased osteoclastogenesis and bone destruction, systemic administration of osteoprotegerin reduces tumor-mediated bone destruction and pain associated with bone cancer. Thus, developments of drugs that target inhibition of RANK signaling are potential therapeutics for metabolic bone disorders and cancer.

The cytoplasmic domain of receptor activator of NF-κB interacts with TRAF family members, specifically TRAF1, 2, 3, 5, and 6. Stimulation of RANK activates members of the MAPK family (i.e., JNK, p38, ERK) and IKKs, which lead to activation of transcription factors AP1 and NF-κB. The interactions of TRAF2, TRAF5, and TRAF6 with receptor activator of NF-κB have been reported and it was demonstrated that receptor activator of NF-κB could activate both the NF-κB and JNK pathways. Subsequently, a novel TRAF6 binding motif was identified in receptor activator of NF-κB that is distinct from the TRAF2 and TRAF5 binding domains.

The TRAF6 binding domain in RANK was sufficient for activation of NF-κB, suggesting that TRAF2 and TRAF5 are not necessary for NF-κB activation. In support of these findings, TRAF6-deficient mice develop osteopetrosis due to a defect in osteoclastogenesis, which is not found in either the TRAF2- or TRAF5-deficient mice.

Since TRAF6 appears to be the critical adapter protein for RANK signaling, the present invention develops a novel TRAF6 decoy peptide (T6DP) with and without a peptide leader sequence that allows for transversing cellular membrane. Evidences disclosed herein indicate that the TRAF6 decoy peptide inhibits RANKL signaling transduction and RANKL-mediated osteoclast differentiation, but only when the leader sequence is attached. These data indicate that targeted disruption of the interaction between receptor activator of NF-κB and TRAF6 may prove useful as a therapeutic for metabolic bone disorders, leukemia, multiple myeloma, arthritis, and metastatic cancer of the bone.

The present invention is drawn to decoy polypeptides that inhibit signaling mediated by TNF receptor-associated factor 6 (TRAF6). These polypeptides comprise a TRAF6 binding domain and a leader signal sequence. A number of approaches may be utilized by a person having ordinary skill in this art to search for TRAF6 decoy polypeptides. For example, two representative approaches are screening peptide libraries or synthesizing overlapping peptides from the cytoplasmic domains of RANK or TRAF6. In one embodiment of the present invention, the decoy polypeptide comprises of sequence selected from the group consisting of SEQ ID No. 19 and 20. Since TRAF6 also mediates signaling induced by a number of molecules such as IL-1, LPS, IL-18, and CD40L, the decoy polypeptides claimed herein may inhibit RANKL mediated signaling as well as signaling induced by these other molecules.

The decoy polypeptides disclosed herein may contain a TRAF6 binding domain derived from CD40, Receptor Activator of NF-κB, IL-1 receptor-associated kinase 1 (IRAK1), IL-1 receptor-associated kinase 2 (IRAK2), IRAK-M or RIP2. Preferably, the TRAF6 binding domain comprises of sequence selected from the group consisting of SEQ ID No. 1-18.

The leader signal sequence attached to the TRAF6 binding domain in the decoy polypeptides disclosed herein may be derived from a number of different proteins. Representative leader signal peptides include Kaposi fibroblast growth factor signal sequence, HIV-1 Tat (48-60), D-amino acid-substituted HIV-1 Tat (48-60), arginine-substituted HIV-1 Tat (48-60), Drosophila Antennapaedia (43-58), viral RNA binding peptide that comprises 7 or more arginines, DNA binding peptide that comprises 7 or more arginines and polyarginine polypeptide that has 6 to 8 arginines. Arginine-rich signal sequences that can be used for delivery of exogenous proteins into cells are well known in the art. For example, Futaki et al. (2001) has reported various arginine-rich peptides that have translocation activities very similar to that of HIV-1 Tat (48-60). These arginine-rich peptides include HIV-1 Rev (34-50), HTLV-II Rev (4-16), brome mosaic virus Gag (7-25), flock house virus coat protein (35-49), human c-Fos (139-164), human c-Jun (252-279) and yeast transcription factor GCN4 (231-252).

The present invention also provides methods of using the decoy polypeptides disclosed herein to inhibit Receptor Activator of NF-κB Ligand (RANKL)-induced osteoclast differentiation. Inhibiting the interaction between RANK and TRAF6 by the decoy polypeptides would result in inhibition of RANKL- or breast cancer cells-induced osteoclast differentiation. The decoy polypeptides can also be used to inhibit breast cancer-induced osteolytic lesions. The decoy polypeptides can be applied to an individual or cells by a number of methods well known in the art such as liposomes, viruses or other gene delivery vectors. For example, the ProVectin™ protein delivery reagent is a unique lipid-based formulation that allows delivery of the decoy polypeptides disclosed herein or other bioactive molecules into a broad range of cell types.

The present invention also provides a method of inhibiting cancer cells-induced osteolytic lesions, comprising the step of administering the composition of the present invention to an individual. Representative means by which the composition is delivered to said individual include liposomes, a virus and a gene delivery vector.

The present invention also provides a method of identifying a non-peptide small molecule capable of inhibiting interaction between receptor activator of NF-κB (RANK) and TNF receptor-associated factor 6 (TRAF6), comprising the step of: preparing a polypeptide comprising a TRAF6 binding domain; and examining binding of TRAF6 to said polypeptide in the presence and absence of a non-peptide small molecule, wherein reduced binding in the presence of said non-peptide small molecule would indicate that said non-peptide small molecule is capable of inhibiting RANK-TRAF6 interaction. Preferably, the TRAF6 binding domain is derived from a protein selected from the group consisting of CD40, Receptor Activator of NF-κB, IL-1 receptor-associated kinase 1 (IRAK1), IL-1 receptor-associated kinase 2 (IRAK2), IRAK-M and RIP2. In one embodiment, the TRAF6 binding domain comprises a sequence selected from the group consisting of SEQ ID NOs: 1-18. In one embodiment of this method, the polypeptide is immobilized on an ELISA microtiter plate. The binding of TRAF6 to said polypeptide is determined by levels of fluorescent activities.

The present invention also provides a pharmaceutical composition comprising a pharmaceutically acceptable carrier and the decoy polypeptides disclosed herein. More specifically, in one embodiment, this pharmaceutical composition comprises TRAF6 binding domain having a sequence selected from the group consisting of SEQ ID NOs: 1-18. More preferably, the polypeptide comprises a sequence selected from the group consisting of SEQ ID NOs: 19 and 20. The phrase “pharmaceutically acceptable” refers to molecular entities and compositions that do not produce an allergic or similar untoward reaction when administered to a subject. The preparation of an aqueous composition that contains a protein as an active ingredient is well understood in the art.

A person having ordinary skill in this art would readily be able to determine, without undue experimentation, the appropriate dosages and routes of administration of the active component of the present invention. When used in vivo for therapy, the active composition(s) of the present invention is administered to the patient or an animal in therapeutically effective amounts, i.e., amounts that inhibit RANKL-mediated osteoclast differentiation or cancer cells-induced osteolytic lesions. See Remington's Pharmaceutical Science, 17th Ed. (1990) Mark Publishing Co., Easton, Penn.; and Goodman and Gilman's: The Pharmacological Basis of Therapeutics 8th Ed (1990) Pergamon Press.

The present invention is further drawn to non-peptide analogs that mimic the functions of the decoy polypeptides disclosed herein, wherein these non-peptide analogs inhibit signaling mediated by TRAF6. Low molecular weight, non-peptide molecules that mimic the inhibitory polypeptides disclosed herein can serve as robust tools to help establish the role of TRAF6-mediated signaling in models of physiological and pathophysiological processes as well as serving as therapeutic agents in their own right. A number of reports have disclosed the rationale and strategy for the design of low molecular weight, non-peptide molecules that are amenable to high resolution analysis and rapid modification (Horwell, 1995; Saragovi et al., 1992; Wexler et al., 1992).

The present invention is also drawn to a method of inhibiting Receptor Activator of NF-κB Ligand (RANKL)-induced osteoclast differentiation using these non-peptide analogs, wherein inhibition of interaction between RANK and TRAF6 by said non-peptide analogs result in inhibition of RANKL-induced osteoclast differentiation. This method can be used to inhibit osteoclast differentiation induced by breast cancer cells.

The following examples are given for the purpose of illustrating various embodiments of the invention and are not meant to limit the present invention in any fashion. The present examples, along with the methods, procedures, treatments, molecules, and specific compounds described herein are presently representative of preferred embodiments. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

Reagents, Cell Lines, and Antibodies The human embryonic kidney 293 cell line and the mouse macrophage cell line RAW264.7 were obtained from the American Type Culture Collection (Rockville, Md.). The 293 cells were cultured in MEM supplemented with 10% fetal bovine serum and antibiotics. RAW264.7 cells were cultured in DMEM-F12 supplemented with 10% fetal bovine serum and antibiotics. Monoclonal antibodies to phospho-ERK, p38, and JNK were purchased from New England Biolabs. Goat anti-rabbit IgG-conjugated horseradish peroxidase was purchased from BioRad Laboratories (Hercules, Calif.). Anti-JNK1 and anti-IκB were purchased from Santa Cruz Biotechnology (Santa Cruz, Calif.). Goat anti-mouse IgG-conjugated horseradish peroxidase was purchased from Transduction Laboratories (Lexington, Ky.). Protein A/G Sepharose beads was purchased from Pierce (Rockford, Ill.), and anti-FLAG was purchased from Sigma (St. Louis, Mo.). Staining for tartrate resistance acid phosphatase (TRAP) positive osteoclasts was performed essentially as described (30) or by using an acid phosphatase kit from Sigma.

### EXAMPLE 2

Expression Plasmids

Expression plasmids encoding mouse FLAG-tagged TRAF5 and TRAF6 (Akiba et al., 1998) were provided by H. Nakano (Juntendo University, Tokyo, Japan) and FLAG-tagged TRAF2 was provided by J. Ni (Human Genome Sciences, Inc.). Expression vectors and purification of GST-fusion proteins for GST, GST-RANK cytoplasmic domain, and GST-Jun (1-79) have been previously described (Darnay et al., 1998, 1999). The expression vector of full-length murine RANKL (also known as TNF-related activation-induced cytokine (TRANCE) (pcDNA3.1-TRANCE) was provided by Y. Choi (Rockefeller University, New York, N.Y.).

To generate a bacterial expression vector for RANKL, specific 5′ and 3′ primers with HindIII and NotI sites, respectively, were used to amplify the DNA which encodes residues 157-316 of RANKL from the pcDNA3-TRANCE template. The PCR product was digested with HindIII/NotI and ligated in-frame with a HA-tag (N-terminal) and a histidine tag (C-terminal) into the expression vector pHB6 (Boerhinger Mannheim). Soluble RANKL was expressed and purified using Ni-agarose.

### EXAMPLE 3

Transient Transfections and Western Blotting

293 cells were plated at 0.6×106 cells/well on 6-well plates and transfected the next day as described (Darnay et al., 1999). Total amount of plasmid DNA was kept constant by adding empty pCMVFLAG1 vector. Cells and the conditioned supernatants were harvested 24-36 h after transfection. Lysates were prepared as described (Darnay et al., 1999). For western blot analysis, whole-cell lysates (15-30 pg) or proteins from GST-affinity precipitation were separated by 8.5% SDS-PAGE, electroblotted onto nitrocellulose membranes, and incubated with the indicated antibodies. The membranes were then developed using the enhanced chemiluminescence (ECL) system (Amersham, Chicago, Ill.).

### EXAMPLE 4

In vitro Osteoclast Differentiation

Primary bone marrow monocytes (BM) or RAW264.7 cells were cultured in 48-well dishes at a density of 1×105 cells/well or 2×103 cells/well, and then treated with the indicated factors at the beginning of the culture and during a medium change on day 3. Osteoclast formation was assessed by counting the total number of multi-nucleated (>3 nuclei), TRAP-positive cells present per well between day 7 and 10 (BM) or on day 5 (RAW264.7).

### EXAMPLE 5

GST-RANK Fusion Protein Affinity Binding Assays

Equivalent amounts of GST or GST-RANK cytoplasmic domain (GST-RANKcd) fusion protein attached to 20 μl of glutathione agarose beads were mixed with lysates (50 μg) from 293 cells programmed to express the epitope-tagged TRAF protein and the indicated peptides in binding buffer (20 mM TRIS, pH 8, 150 mM NaCl, 1 mM DTT, 2 mM EDTA, and 0.1% NP-40) and allowed to rotate for 1 h at 40C. The beads were collected by centrifugation, washed three times in binding buffer, and then washed once in low-salt buffer (20 mM TRIS, pH 8, 50 mM NaCl, and 1 mM DTT). Bound proteins were eluted with addition of SDS-sample buffer and boiled. The eluted proteins were subjected to 7.5% SDS-PAGE and western blot analysis was performed with anti-FLAG antibodies.

### EXAMPLE 6

Immune Complex Kinase Assays

Lysates were prepared from RAW cells stimulated with RANKL as indicated in the legends to the figures. Approximately 30 μg was then used for immunoprecipitation with indicated antibodies and protein A/G Sepharose beads for 1 h. Beads were collected by centrifugation, washed three times in lysis buffer, and then washed two times in low-salt buffer. JNK activity was analyzed using exogenous GST-Jun (1-79) as a substrate as previously described (Darnay et al., 1999). Kinase activity was quantitated using a PhosphoImager and Imagequant Software (Molecular Dynamics, Sunnyvale, Calif.).

### EXAMPLE 7

Electrophoretic Mobility Shift Assays (EMSA)

Nuclear extracts were prepared from transfected cells essentially as described (Haridas et al., 1998). Equivalent amounts of nuclear protein were used in an EMSA reaction with ³²P-labeled NF-κB oligonucleotide from HIV-LTR as described (Haridas et al., 1998). NF-κB activation was quantitated using a PhosphoImager and Imagequant Software.

### EXAMPLE 8

TRAF6 Binding Domain

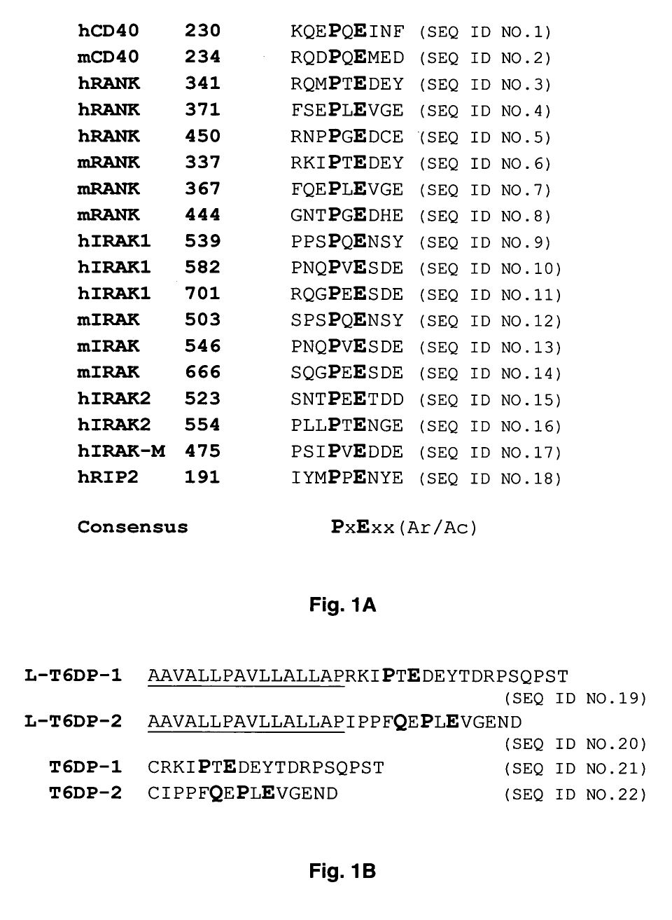

A novel TRAF6 binding domain in RANK, which binds to only TRAF6 but not TRAF2 or TRAF5, has been identified previously (Darnay et al., 1999). When transfected in 293 cells, this region of RANK was sufficient for activation of NF-κB (Darnay et al., 1999). Structure-based sequence alignment of TRAF6-binding sites in human and mouse CD40 and RANK led to the definition of a TRAF6-binding motif PxExx(Ar/Ac) (Ar for aromatic and Ac for acidic residues) (FIG. 1A). Careful examination of the RANK sequence indicates that there are three potential TRAF6-binding sites (FIG. 1A).

TRAF6 is the only TRAF family member that participates in the signal transduction of both the TNF receptor superfamily and the interleukin-1 receptor (IL-1R)/Toll-like receptor (TLR) superfamily. The best-characterized TRAF6 signaling pathway for the IL-1R/TLR superfamily involves IRAK, an adapter kinase upstream of TRAF6. Upon receptor stimulation, IRAK becomes oligomerized and interacts with TRAF6. It was found that full-length IRAK contains three potential TRAF6-binding sites (FIG. 1A). Two IRAK homologues, IRAK-2 and IRAK-M, also contain two and a single potential TRAF6-binding site, respectively (FIG. 1A). This is in keeping with the implicated role of IRAK-2 and IRAK-M in IL-1 signaling and the role of IRAK-2 in TLR4 signaling. In addition, it was found that the kinase RIP2, which can activate NF-κB and induce cell death, also contains a putative TRAF6-binding site (FIG. 1A). The presence of TRAF6-binding sites in these various molecules suggests that TRAF6 may play a role in mediating multiple signaling cascades.

Delivery of peptides or proteins across cellular membranes can be achieved by covalent attachment of the peptide to molecules that can freely pass through the membrane (Schwarze et al., 2000). For example, the hydrophobic domain of Kaposi fibroblast growth factor signal sequence was attached to the nuclear localization signal from the p50 subunit of NF-κB to allow for translocation across the membrane (Yan et al., 2000). In the present invention, this hydrophobic sequence was attached to peptides derived from murine RANK, namely L-T6DP-1 which contains the known TRAF6 binding domain and L-T6DP-2 which contains a similar motif (Fig. 1B). In addition, both of these peptides were synthesized without the leader sequence, resulting in peptides that were not be able to transverse cellular membrane (FIG. 1B).

### EXAMPLE 9

TRAF6 Decoy Peptides Inhibit RANKL-mediated Osteoclast Differentiation in RAW264.7 Cells

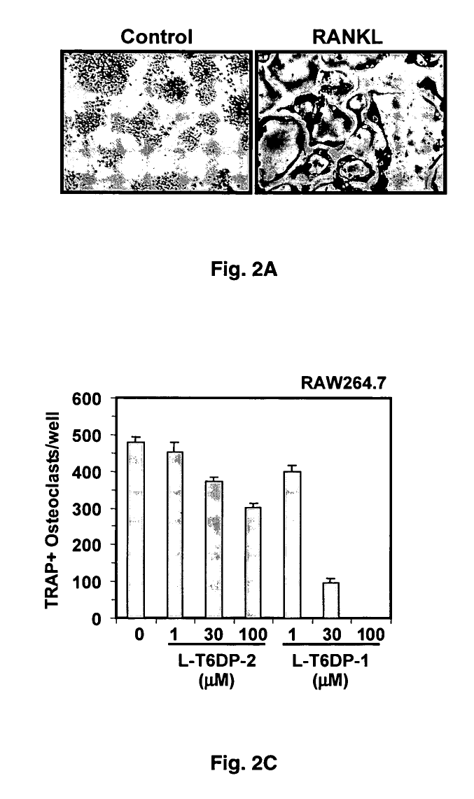

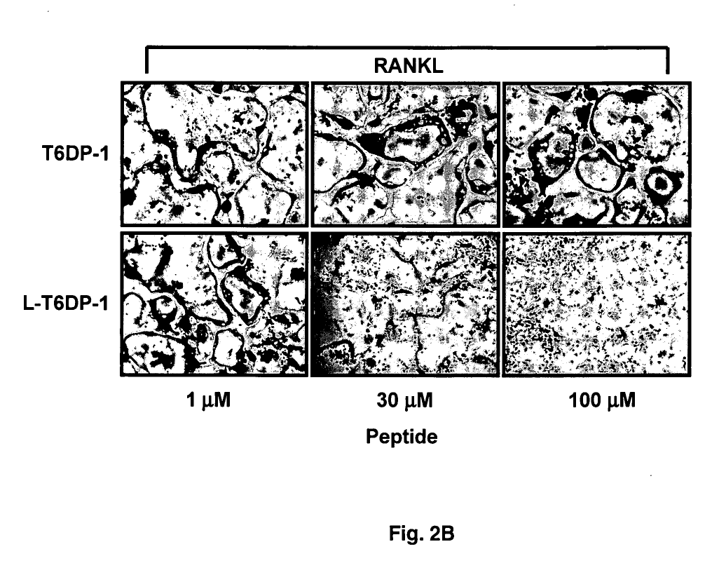

The mouse macrophage cell line RAW264.7 express RANK on their cell surface and when stimulated with RANKL differentiate into multi-nucleated, tartrate resistant acid phosphatase (TRAP) positive osteoclasts after 4-5 days (FIG. 2A). To determine whether L-T6DP-1 could inhibit RANKL-mediated osteoclast differentiation, RAW264.7 cells were co-cultured with increasing concentrations of either L-T6DP-1 or T6DP-1 and 30 ng/ml RANKL for 4 days. As indicated in FIG. 2B, TRAF6 decoy peptide without leader sequence failed to block RANKL-mediated osteoclast differentiation; however, treatment with L-T6DP-1 caused a dose-dependent inhibition of osteoclast differentiation. Although the cells were TRAP positive after treatment with 100 μM L-T6DP-1, multi-nucleated osteoclast were not observed (FIG. 2B). Furthermore, treatment of RAW264.7 cells with RANKL in the presence of either L-T6DP-1 or L-T6DP-2 caused a dose-dependent decrease of TRAP positive osteoclasts (FIG. 2C), although L-T6DP-1 was much more efficient than L-T6DP-2.

### EXAMPLE 10

TRAF6 Decoy Peptides Inhibit RANKL-mediated Osteoclast Differentiation in Bone Marrow-derived Mouse Monocytes

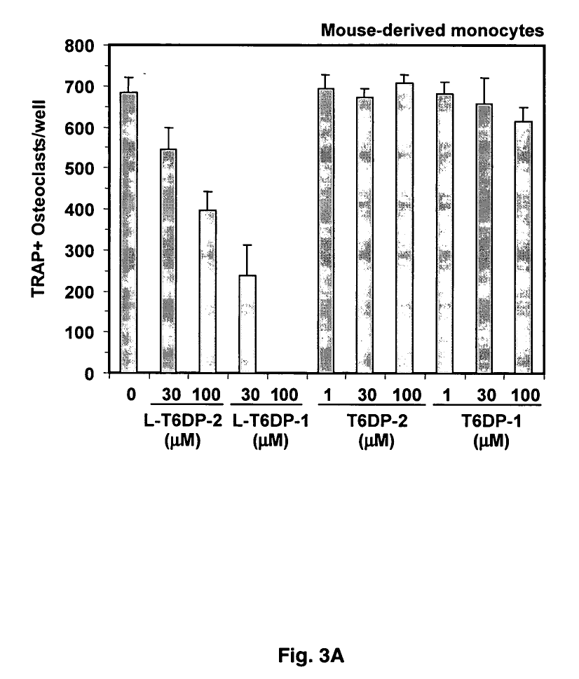

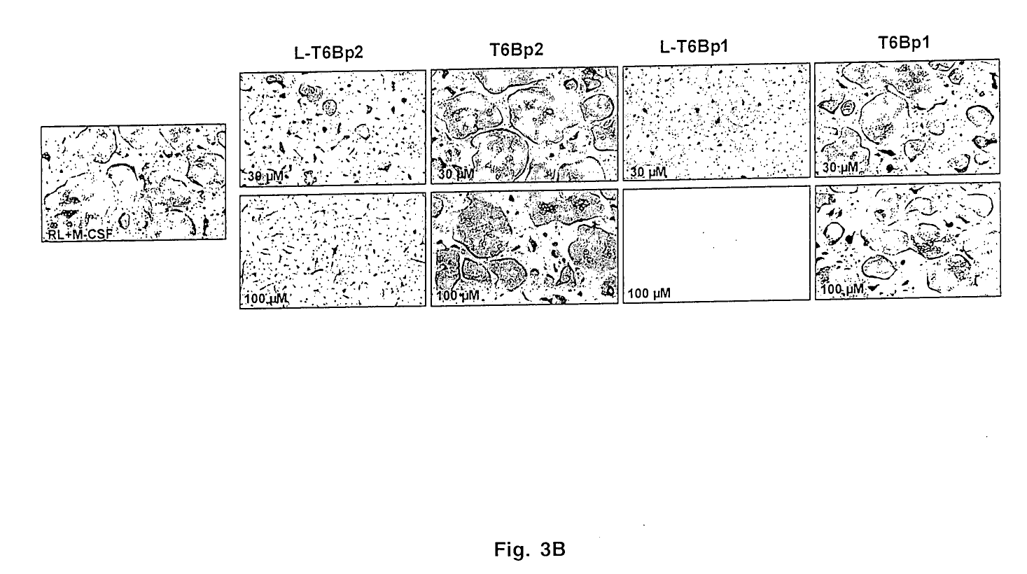

To further support the results obtained from the RAW264.7 cell line, the ability of these peptides to inhibit RANKL-mediated osteoclast differentiation in primary mouse-derived monocytes was tested. Costimulation of bone marrow-derived monocytes with RANKL and M-CSF cause osteoclast differentiation after 7-10 days, as determined by staining multi-nucleated, TRAP positive osteoclasts (Shevde et al., 2000). Similar to the results with RAW264.7 cells, both L-T6DP-1 and L-T6DP-2 inhibited the development of TRAP positive osteoclast in a dose-dependent manner and L-T6DP-1 was much more efficient (FIG. 3). TRAF6 decoy peptides without the leader sequences failed to inhibit osteoclast differentiation (FIG. 3), indicating that the peptides were not toxic to the cells. Although the cells were TRAP positive after treatment with 30 μM L-T6DP-1, multi-nucleated osteoclast were not observed and at 100 μM L-T6DP-1, no osteoclasts were observed (FIG. 3B). Taken together, these results indicate that interaction of TRAF6 with RANK is essential for RANKL-mediated osteoclast differentiation.

### EXAMPLE 11

TRAF Decoy Peptide Inhibits RANKL-mediated NF-kB Activation in RAW264-7 Cells

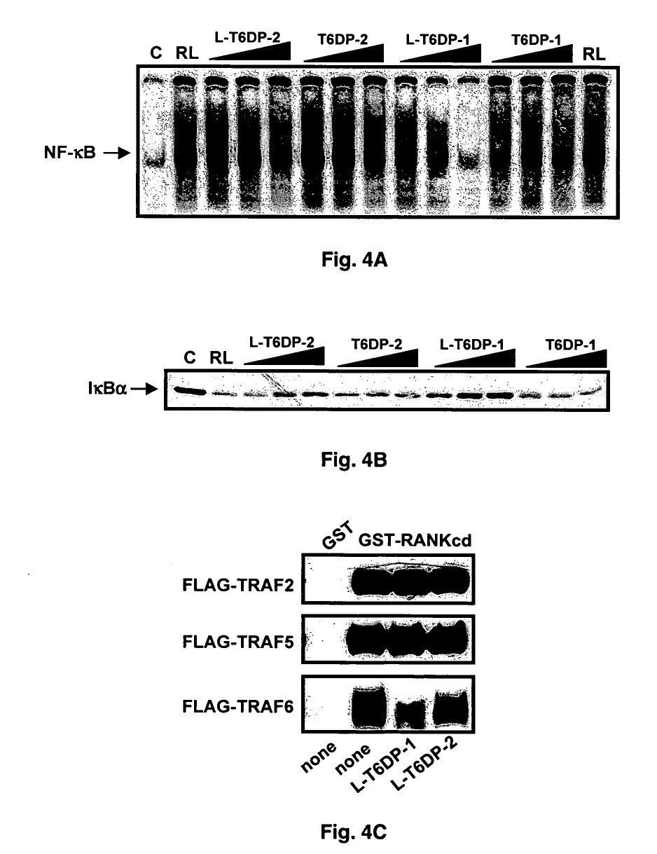

It has been shown that a mutant form of receptor activator of NF-κB which contains only the TRAF6 binding domain is sufficient to activate NF-κB, and a dominant negative TRAF6 inhibits RANK-mediated NF-κB activation in 293 cells. To investigate the effect of the TRAF6 binding peptides on RANKL-induced NF-κB activation, NF-κB activation was examined in RAW264.7 cells that activated NF-κB when stimulated with RANKL. RANKL-stimulated RAW264.7 cells activated NF-κB as indicated by a gel mobility shift assay (FIG. 4A). NF-κB activation was suppressed in a dose-dependent manner only by pre-treatment with L-T6DP-1 (FIG. 4A). The levels of IκBα coincided with the activation and repression of NF-κB as indicated in FIG. 4B. These data indicate that L-T6DP-1 specifically inhibits RANKL-mediated NF-κB activation in RAW264.7 cells.

### EXAMPLE 12

TRAF6 Decoy Peptide Specifically Inhibits TRAF6 Binding to the RANK Cytoplasmic Domain

The cytoplasmic domain of RANK interacts with many TRAF molecules, including TRAF1, 2, 3, 5, and 6. While TRAF1, 2, 3, and 5 interact with the c-terminal tail of receptor activator of NF-κB, TRAF6 interacts with a membrane proximal region of the cytoplasmic domain of RANK. To confirm that the TRAF6 decoy peptides disclosed herein were specifically inhibiting TRAF6 interaction with RANK, a competitive GST-pull down assay was performed.

Cellular extracts containing FLAG-tagged TRAF2, TRAF5, or TRAF6 were mixed with GST-RANK cytoplasmic domain fusion protein in the presence and absence of either L-T6DP-1 or L-T6DP-2. If these peptides competed for the TRAF molecules, less FLAG-tagged protein would be observed in the western blots. As shown in FIG. 4, neither L-T6DP-1 nor L-T6DP-2 inhibited TRAF2 and TRAF5 binding to the cytoplasmic domain of RANK (FIG. 4C). Only L-T6DP-1 inhibited TRAF6 interaction with the cytoplasmic domain of RANK (FIG. 4C). These data indicate that the leader sequence did not interfere with interaction of RANK with TRAFs and that L-T6DP-1 specifically inhibited RANK's interaction with TRAF6.

### EXAMPLE 13

TRAF6 Decoy Peptide Specifically Inhibits JNK, ERK, and p38 Kinase Activation by RANKL in RAW264.7 Cells

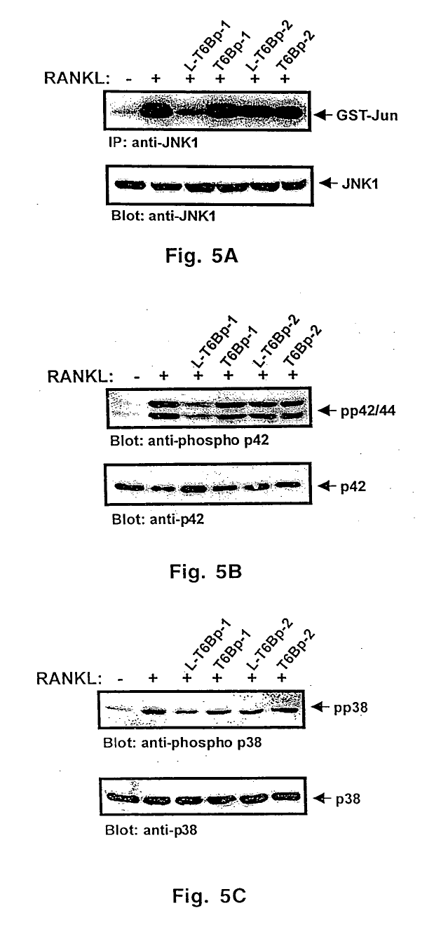

Stimulation of RANK activates members of the MAPK family including JNK, ERK and p38 kinase. The ability of the TRAF6 deocy peptides to inhibit RANKL-induced JNK, ERK, and p38 kinase activation was examined in RAW264.7 cells. In vitro JNK kinase assays indicated JNK is activated by RANKL and only treatment with L-T6DP-1 peptide was capable of inhibiting RANKL-mediated JNK activation (FIG. 5A). Similar to the results with JNK, only L-T6DP-1 was able to block ERK (FIG. 5B) and p38 kinase (FIG. 5C) activation induced by RANKL in RAW264.7 cells. Taken with the results disclosed above, the present invention demonstrates that L-T6DP-1 is able to suppress RANKL-mediated osteoclast differentiation and RANKL-initiated early signaling including NF-κB, JNK, ERK, and p38 kinase activation.

### EXAMPLE 14

Induction of Osteoclast Formation By Breast Cancer Cells and Inhibition by TRAF6 Decoy Peptide

Breast cancer is the most common female malignancy in the U.S. and is the second leading cause of cancer death in women. Women with breast cancer are at risk for bone metastases. Five to ten percent of patients with breast cancer initially present with metastatic disease to the bone. Patients with osteolytic bone disease from metastatic breast cancer are at increased risk for pathologic fractures, bone pain, cord compression and hypercalcemia. Current standard of care for treating bone metastases is bisphosphonate therapy which delays skeletal events but does not completely prevent them. In addition, not all patients respond to this treatment. While a more effective treatment is desired, a further biological and molecular dissection of this disease is required. In fact, recently it was demonstrated that osteoprotegerin (OPG) inhibits osteolysis and decreases tumor burden in nude mouse models injected with breast cancer cells.

The ability of breast cancer cells to induce osteoclast formation and the expression of RANK/RANKL/OPG in breast cancer cells are not well defined. Few reports have demonstrated the ability of breast cancer cell lines to influence osteoclast differentiation and function; however, no evidence has been described for the direct involvement of RANKL in this process. As indicated below, there is evidence to support the hypothesis that breast cancer cells directly induce osteoclast differentiation and function in the absence of osteoblast/stromal cells. Through the understanding of the biological and molecular role of RANKL in breast cancer cells in the bone microenvironment and development of novel inhibitors of osteoclast formation as described herein, alternative therapeutic approaches or combination therapy may be developed to treat breast cancer patients with bone metastases.

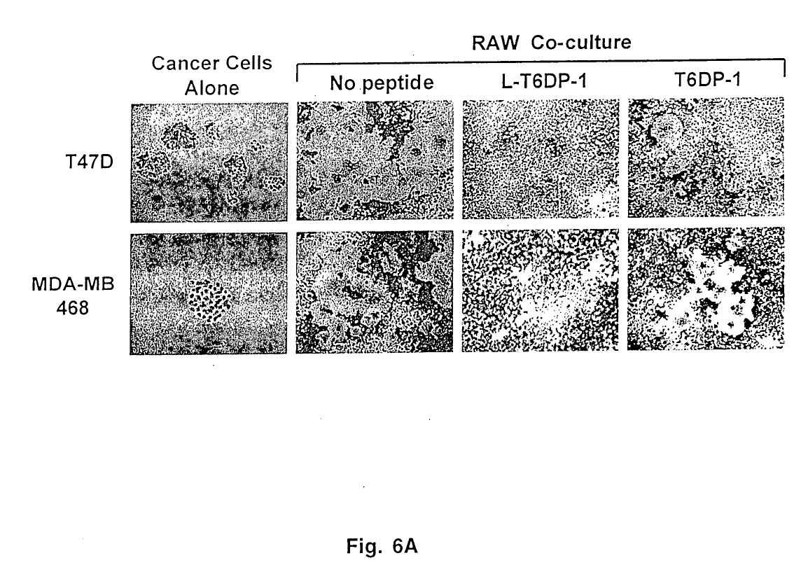

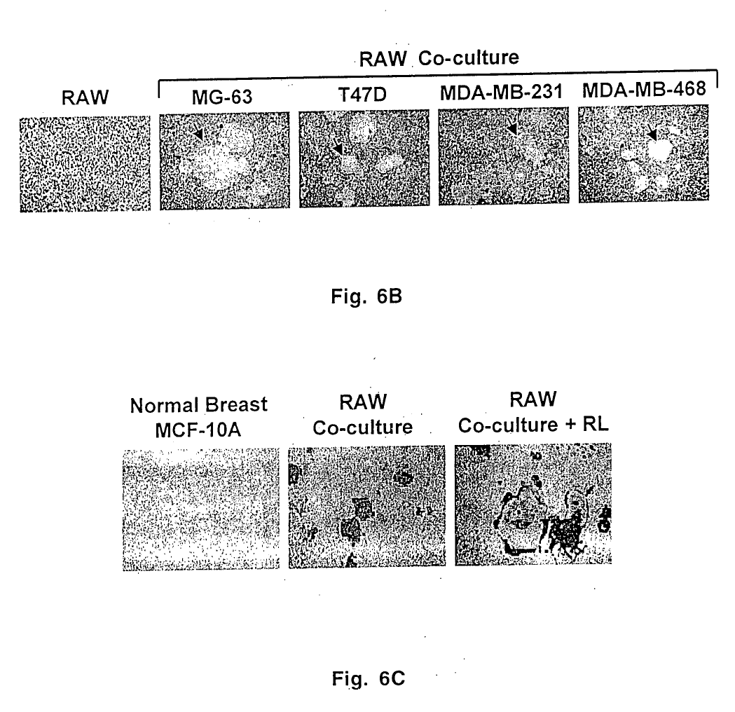

In the present example, a co-culture assay system was developed for RAW cells and the osteoblast-like cell (osteosarcoma MG-63) which has been shown to express RANKL and cause osteoclast differentiation. The number of MG-63 cells was critical for inducing RAW cells to differentiate into osteoclasts. These observations lead to a direct inverse relationship between the number of RAW cells to MG-63 cells which is required for the formation of osteoclasts. Results using RAW cells co-cultured with MG-63 (data not shown) or with breast cancer cell lines (i.e., T47D and MDA-MB-468) indicated that these breast cancer cells could in fact cause RAW cells to form TRAP+, multi-nucleated osteoclasts after 4 days (FIG. 6A) similar to RAW cells stimulated with RANKL. In addition, when the breast cancer cell lines were grown in tissue culture inserts where they were separated from RAW cells by a membrane, osteoclast still formed, suggesting that direct cell-to-cell contact was not required for osteoclast differentiation (data not shown). When grown on synthetic bone slides, the osteoclasts derived from the co-culture assays were able to cause bone resorption (FIG. 6B).

When L-T6DP-1 was added to these co-culture assays, the ability of the breast cancer cells to induce osteoclast differentiation of RAW cells was abolished, whereas T6DP-1 had no effect (FIG. 6A). Normal breast epithelial cells, MCF-10A, failed to induce osteoclast differentiation of RAW; however, osteoclasts did form if exogenous RANKL was added to these co-cultures (FIG. 6C). Collectively, these data indicate that breast cancer cell lines, but not normal breast epithelial cells, can directly induce osteoclast differentiation in the absence of osteoblast/stromal cells and that L-T6DP-1 can inhibit this process.

### EXAMPLE 15

Crystal Structure of TRAF6 in Complex with Binding Peptides

The unique biological function of TRAF6 is largely determined by its TRAF-C domain, which does not interact with peptide motifs recognized by TRAF1, 2, 3 and 5. To elucidate the molecular basis of this specificity, crystal structures were determined for the TRAF-C domain of TRAF6 (residues 346-504), alone and in complex with a peptide from human CD40 (residues 230-238) or RANK/TRANCE-R (residues 342-349) (Ye et al., 2002).

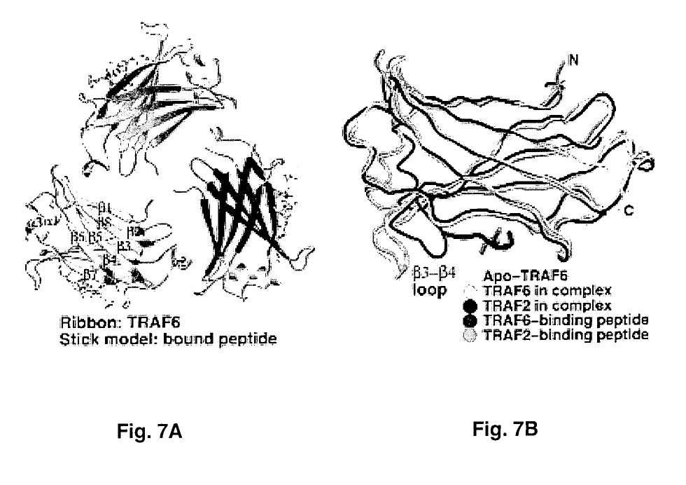

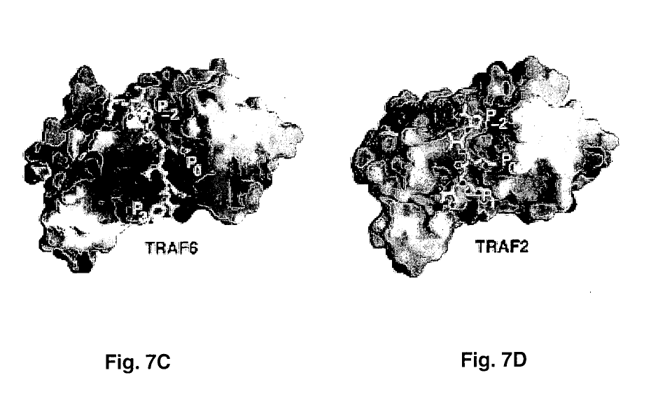

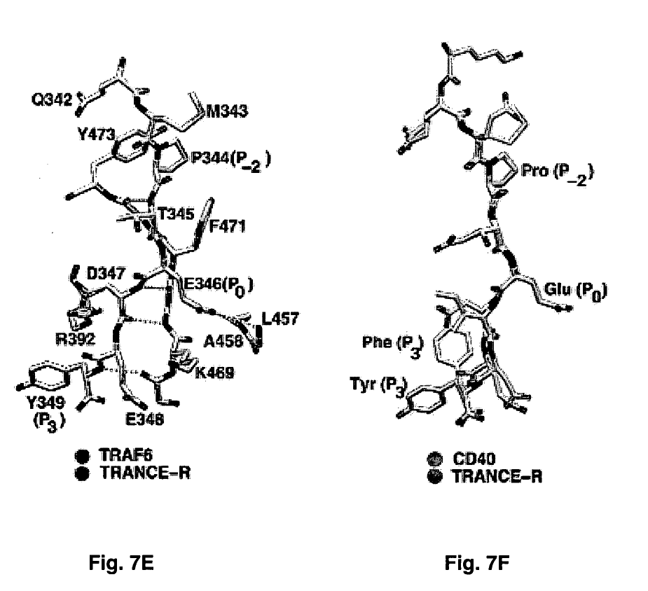

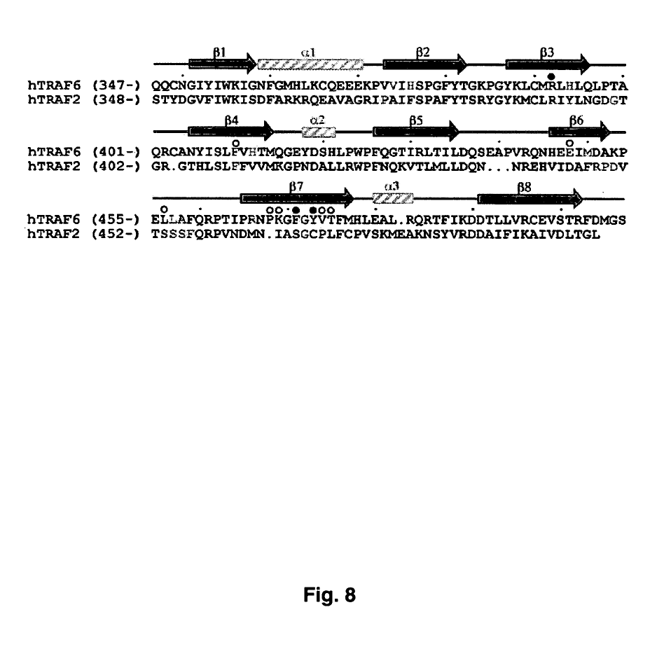

Surprisingly, there are striking differences in the binding mode of the peptides to TRAF6 as compared to the binding modes of peptides to other TRAFs. First, the chain direction of bound TRAF6-binding peptides exhibits a 400 difference to that of TRAF2-binding peptides (FIGS. 7B-D). As a result, the side chains of TRAF6-binding peptides interact with binding pockets that are completely different from those in peptide recognition by TRAF2. Second, the TRAF6-binding peptides assume extended β-conformations, rather than the poly-proline II (PPII) helix conformation for the core region of TRAF2-binding peptides (FIG. 7B). In addition, the peptides make more extensive main chain hydrogen bonds with the β7 strand of the TRAF-C domain (residues 234-238 of CD40 and 344-349 of RANK with residues Pro 468-Gly 472 of TRAF6), which is facilitated by the insertion of Pro 468 in the β-bulge of this strand (FIG. 7E). Finally, the peptides no longer interact with the β3-β4 loop in the TRAF6 complexes due to a 12 Å movement in the position of this loop (FIG. 7B).

### EXAMPLE 16

In vivo Mouse Model of Breast Cancer-Induced Osteolysis

Recently, osteoprotegerin (OPG) administration in nude mouse models of experimental bone metastasis resulted in decreased osteolysis and skeletal tumor burden. Furthermore, preliminary data from a phase I clinical trial of osteoprotegerin treatment to women with breast cancer bone metastasis showed a 60-70% decrease in bone resorption. Although osteoprotegerin had no adverse side effects in the patients, osteoprotegerin was immunogenic; and thus, further experimentation is evident.

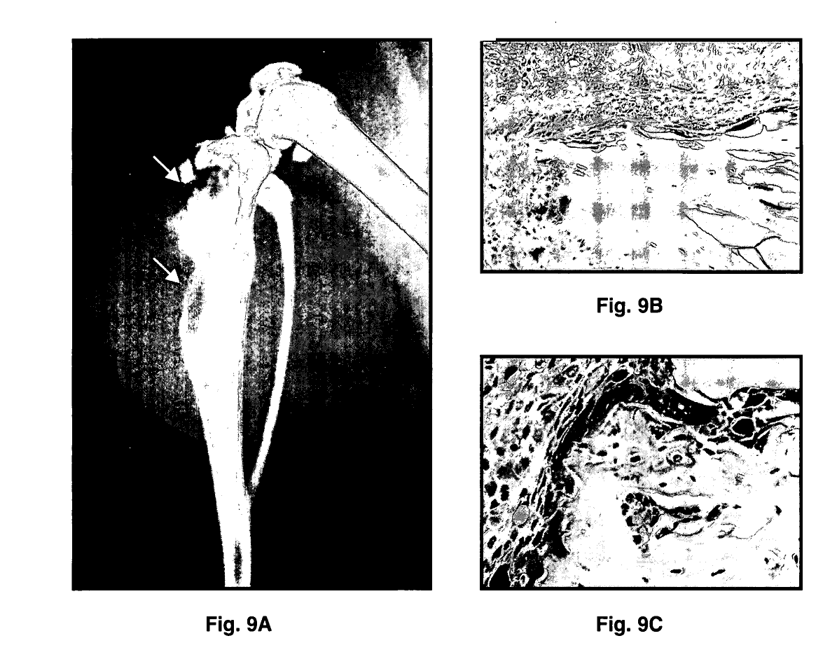

As an alternative approach to examine potential inhibitors of bone destruction associated with bone metastasis, an in vivo mouse model was developed in which the breast cancer cells lines MDA231 or SUM 149 were injected into the proximal tibia of nude mice. This injection route was used to assess the ability of tumor cells to grow in the bone and to form osteolytic lesions after approximately 4 weeks as indicated by radiographic imaging of the bone (FIG. 9A). Furthermore, sections of the tumor-bone interface can be stained for TRAP⁺osteoclasts, indicating an increase of osteoclastic activity (FIGS. 9B-C). This model system can be used to examine the ability of TRAF6 decoy peptides to inhibit osteolytic lesions induced by breast cancer tumor cells.

### EXAMPLE 17

Treatment of Breast Cancer Bone Tumors With TRAF6 Decoy Peptide

In an effort to evaluate whether T6DPs could inhibit breast cancer-induced osteolytic lesions in vivo, T6DPs was examined in the SUM149 breast cancer model described above. Nude mice were injected with SUM149it1 breast cancer cells. The SUM149it1 cell line is a variant of the SUM149PT cell line obtained from the University of Michigan Cancer Center. This variant was developed at the University of Michigan from a bone tumor in a nude mouse injected with the original cell line, and showed more rapid development of osteolytic tumors than the non-selected cell line.

Suspensions of cells (1-5×10⁵ in 0.02 ml of PBS) were injected through the cortex of anterior tuberosity of the tibia of nude mice. This injection route is used to assess the ability of tumor cells to grow in the bone and to form osteolytic lesions. The mice were anesthetized with isoflurane (2%). The hind leg was cleaned with betadyne and 70% ethanol. The needle (26 G) was inserted through the cortex of anterior tuberosity of the tibia, rotating the needle to minimize cortical fracture. Once through the cortex, the needle was inserted 3 mm further down the diaphysis of the tibia, and 0.02 ml of cell suspension in sterile saline was injected. The dose of injected cells has previously been shown to form lytic tumors in 100% of animals.

Beginning on day three after injection, the mice received i.p. injection 3 times per week of either 0.1 ml PBS, or 1 mg of the L-T6DP. The mice were weighed weekly, and X-ray images of the tibias were taken on day 28 after injection and at 2-week intervals thereafter.

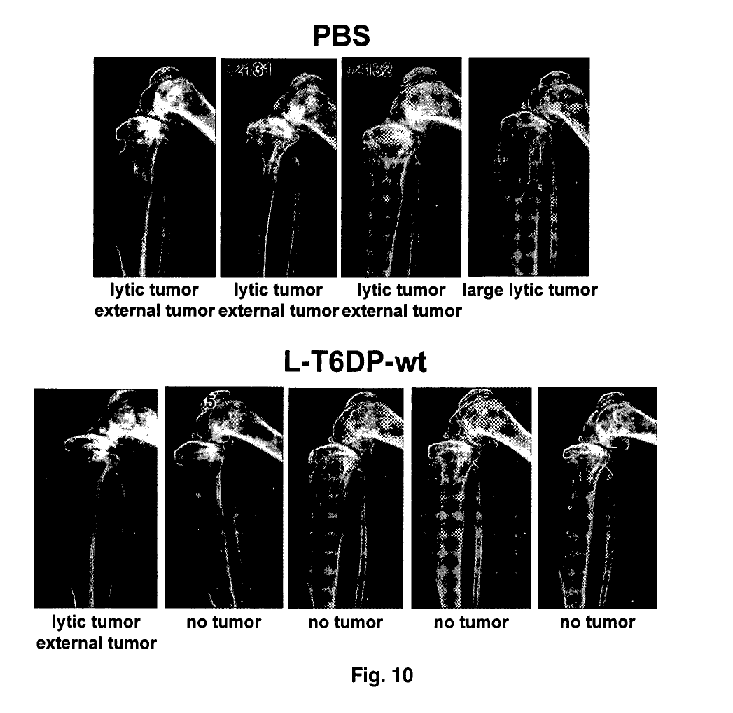

The injections of the peptides, 3 times per week, were tolerated, and there was no weight loss or other signs of toxicity in the treated mice. The mice received 29 injections over a period of 10 weeks. Results from X-ray and histology examination indicated that in the PBS group, there were 4 out of 4 tumor growth in bone, one with extensive bone lysis (FIG. 10). In the L-TR6DP treatment group, 1 out of 5 mice had tumor growth (with lysis) in bone; 4 out of 5 mice had no tumor (FIG. 10). Decalcified sections were stained for presence of TRAP in activated osteoclasts. There were no obvious differences in the numbers of TRAP⁺cells in any of the sections (in areas close to or away from tumor), regardless of the treatment group.

### EXAMPLE 18

Mutation of TRAF6 Decoy Peptide

L-T6DP-mut is a mutant form in which the three conserved residues in the TRAF6 binding sites at the P₋₂, P₀, and P₃ postions have been mutated to alanine residues. The peptide is provided by Dr. Sujay Singh (Imgenex, Inc, San Diego, Calif.) and synthesized using an automated peptide synthesizer (Symphony, Rainin Instrument Co., Inc., Woburn, Mass.). The peptide is purified by HPLC and the molecular weight is analyzed by Mass Spectrometry.

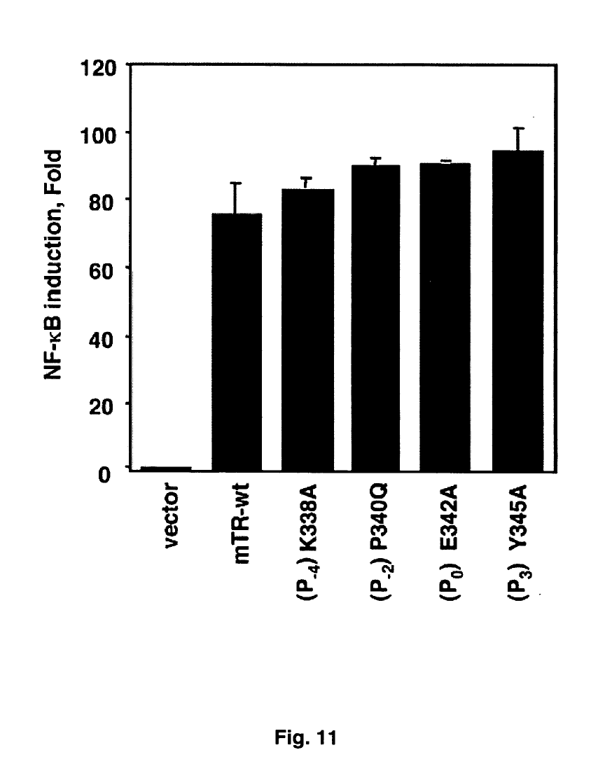

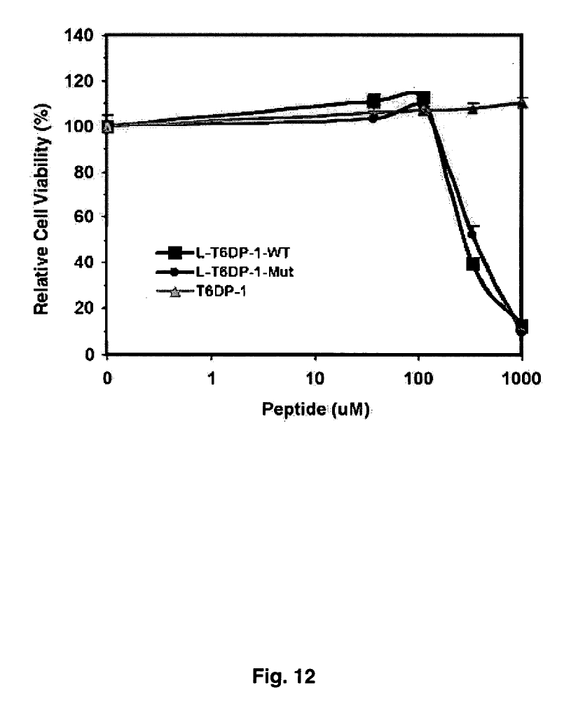

Mutations at P-2, P₀ and P3 positions of a TRAF6-binding site in full-length TRANCE-R did not affect NF-kB activation (FIG. 11). FIG. 12 shows that the triple alanine mutant of L-T6DP-1 has the same capacity as wild type L-T6DP-1 in inhibiting the growth of RAW264.7 cells.

### EXAMPLE 19

Strategy to Discover Non-peptide Analogues that Inhibit RANK-TRAF6 Interaction

An ELISA-based method similar to one described previously (J. Biol. Chem. (2001), 276:12235-12240) can be used to discover small molecules that inhibit RANK-TRAF6 interaction. Briefly, peptides comprising the T6DP are biotinylated, dissolved in TRIS-buffered saline (50 mM TRIS pH 7.5, 150 mM NaCl), and added to wells in NeutrAvidin-coated 96-well microtiter plates. The plates are shaken overnight at 40° C. and then rinsed with TBS followed by TBS-BT (TBS containing 0.1% bovine serum albumin (BSA) and 0.1% Tween 20). A solution containing a test small molecule from a library is then added to the well. A solution containing 6×-histidine-tagged TRAF6 (309-522) is then added to each well. The plates are incubated for 1 h at room temperature and then washed 3 times with TBS-BT. An antibody directed against the C-terminus of TRAF6 is then added to the wells and the plates are incubated for 1 hour at room temperature, followed by 3 washes with TBS-BT. A secondary antibody consisting of goat anti-mouse alkaline phosphatase is then added to each well and the plates are incubated for 1 hour at room temperature, followed by 3 washes with TBS-BT. The plates are then assayed for alkaline phosphatase activity using a fluorescent plate reader as previously described (Darnay et al., 1999).

The following references were cited herein:

Akiba et al., CD27, a member of the tumor necrosis factor receptor superfamily, activates NF-kappaB and stress-activated protein kinase/c-Jun N-terminal kinase via TRAF2, TRAF5, and NF-kappaBinducing kinase. J Biol. Chem. 273:13353 (1998).

Darnay et al., Characterization of the intracellular domain of receptor activator of NF-kB (RANK). Interaction with tumor necrosis factor receptor-associated factors and activation of NF-kB and c-Jun N-terminal kinase. J Biol. Chem. 273:20551 (1998).

Darnay et al., Activation of NF-kB by RANK requires tumor necrosis factor receptor-associated factor (TRAF) 6 and NF-kB-inducing kinase. Identification of a novel TRAF6 interaction motif. J Biol. Chem. 274:7724 (1999).

Futaki et al., Arginine-rich Peptides. J Biol. Chem. 276:5836 (2001).

Haridas et al., Overexpression of the p80 TNF receptor leads to TNF-dependent apoptosis, nuclear factor-kappa B activation, and c-Jun kinase activation. J Immunol. 160:3152 (1998).

Horwell. Trends Biotechnol. 13:132 (1995).

Pullen et al., CD40 signaling through tumor necrosis factor receptor-associated factors (TRAFs). Binding site specificity and activation of downstream pathways by distinct TRAFs. J Biol. Chem. 274:14246 (1999).

Saragovi et al. Biotechnology 10:773 (1992).

Schwarze et al. Protein transduction: unrestricted delivery into all cells? Trends Cell Biol. 10:290 (2000).

Shevde et al., Estrogens suppress RANK ligand-induced osteoclast differentiation via a stromal cell independent mechanism involving c-jun repression. Proc. Natl. Acad. Sci. USA 97:7829 (2000).

Wexler et al. Am. J. Hypertens. 5:209S (1992).

Yan et al., Peptide-directed suppression of a pro-inflammatory cytokine response. J Biol. Chem. 275:16774 (2000).

Ye et al., Distinct molecular mechanism for initiating TRAF6 signaling. Nature 418:443 (2002).

Any patents or publications mentioned in this specification are indicative of the levels of those skilled in the art to which the invention pertains. Further, these patents and publications are incorporated by reference herein to the same extent as if each individual publication is specifically and individually indicated to be incorporated by reference.

What is claimed is:

1. A polypeptide that inhibits signaling mediated by TNF receptor-associated factor 6 (TRAF6), wherein said polypeptide comprises a TRAF6 binding domain and a leader signal sequence.

2. The polypeptide of claim 1, wherein said leader signal sequence comprises a polypeptide selected from the group consisting of Kaposi fibroblast growth factor signal sequence, HIV-1 Tat (48-60), D-amino acid-substituted HIV-1 Tat (48-60), arginine-substituted HIV-1 Tat (48-60), Drosophila Antennapaedia (43-58), viral RNA binding peptide that comprises 7 or more arginines, DNA binding peptide that comprises 7 or more arginines and polyarginine polypeptide that has 6 to 8 arginines.

3. The polypeptide of claim 2, wherein said viral RNA binding peptide is selected from the group consisting of HIV-1 Rev (34-50), HTLV-II Rev (4-16), brome mosaic virus Gag (7-25) and flock house virus coat protein (35-49).

4. The polypeptide of claim 2, wherein said DNA binding peptide is selected from the group consisting of human c-Fos (139-164), human c-Jun (252-279) and yeast transcription factor GCN4 (231-252).

5. The polypeptide of claim 1, wherein said TRAF6 binding domain is a TRAF6 binding domain from a protein selected from the group consisting of CD40, Receptor Activator of NF-κB, IL-1 receptor-associated kinase 1 (IRAK1), IL-1 receptor-associated kinase 2 (IRAK2), IRAK-M and RIP2.

6. The polypeptide of claim 1, wherein said TRAF6 binding domain comprises a sequence selected from the group consisting of SEQ ID NOs: 1-18.

7. The polypeptide of claim 1, wherein said polypeptide comprises a sequence selected from the group consisting of SEQ ID NOs: 19 and 20.

8. A method of inhibiting Receptor Activator of NF-κB Ligand (RANKL)-induced osteoclast differentiation, comprising the step of:
applying the polypeptide of claim 1 to osteoclast, wherein inhibition of interaction between Receptor Activator of NF-κB and TRAF6 by said polypeptide results in inhibition of RANKL-induced osteoclast differentiation.

9. The method of claim 8, wherein said polypeptide is delivered to said cells by a mean selected from the group consisting of liposomes, a virus and a gene delivery vector.

10. The method of claim 8, wherein said osteoclast differentiation is induced by breast cancer cells.

11. A method of inhibiting osteoclast differentiation in an individual, comprising the step of:
applying to said individual the polypeptide of claim 1, wherein inhibition of interaction between Receptor Activator of NF-κB and TRAF6 by said polypeptide results in inhibition of osteoclast differentiation.

12. The method of claim 11, wherein said individual has a disease selected from the group consisting of metabolic bone disorders, leukemia, multiple myeloma, arthritis, and metastatic cancer of the bone.

13. A pharmaceutical composition comprising the polypeptide of claim 1 and a pharmaceutically acceptable carrier.

14. The composition of claim 13, wherein said polypeptide comprises TRAF6 binding domain having a sequence selected from the group consisting of SEQ ID NOs: 1-18.

15. The composition of claim 13, wherein said polypeptide comprises a sequence selected from the group consisting of SEQ ID NOs: 19 and 20.

16. A method of inhibiting cancer cells-induced osteolytic lesions, comprising the step of administering the composition of claim 13 to an individual.

17. The method of claim 16, wherein said composition is delivered to said individual by a mean selected from the group consisting of liposomes, a virus and a gene delivery vector.

18. The method of claim 16, wherein said cancer cells are breast cancer cells or prostate cancer cells.

19. A method of identifying a non-peptide small molecule capable of inhibiting interaction between receptor activator of NF-κB (RANK) and TNF receptor-associated factor 6 (TRAF6), comprising the step of:
preparing a polypeptide comprising a TRAF6 binding domain; and
examining binding of TRAF6 to said polypeptide in the presence and absence of a non-peptide small molecule, wherein reduced binding in the presence of said non-peptide small molecule would indicate that said non-peptide small molecule is capable of inhibiting RANK-TRAF6 interaction.

20. The method of claim 19, wherein said TRAF6 binding domain is derived from a protein selected from the group consisting of CD40, Receptor Activator of NF-κB, IL-1 receptor-associated kinase 1 (IRAK1), IL-1 receptor-associated kinase 2 (IRAK2), IRAK-M and RIP2.

21. The method of claim 20, wherein said TRAF6 binding domain comprises a sequence selected from the group consisting of SEQ ID NOs: 1-18.

22. The method of claim 19, wherein said polypeptide is immobilized on an ELISA microtiter plate.

23. The method of claim 19, wherein binding of TRAF6 to said polypeptide is determined by levels of fluorescent activities.

24. A non-peptide analog that mimics the function of a polypeptide comprising a TNF receptor-associated factor 6 (TRAF6) binding domain and a leader signal sequence, wherein said polypeptide inhibits signaling mediated by TRAF6.

25. The non-peptide analog of claim 24, wherein said leader signal sequence comprises a polypeptide selected from the group consisting of Kaposi fibroblast growth factor signal sequence, HIV-1 Tat (48-60), D-amino acid-substituted HIV-1 Tat (48-60), arginine-substituted HIV-1 Tat (48-60), Drosophila Antennapaedia (43-58), viral RNA binding peptide that comprises 7 or more arginines, DNA binding peptide that comprises 7 or more arginines and polyarginine polypeptide that has 6 to 8 arginines.

26. The non-peptide analog of claim 25, wherein said viral RNA binding peptide is selected from the group consisting of HIV-1 Rev (34-50), HTLV-II Rev (4-16), brome mosaic virus Gag (7-25) and flock house virus coat protein (35-49).

27. The non-peptide analog of claim 25, wherein said DNA binding peptide is selected from the group consisting of human c-Fos (139-164), human c-Jun (252-279) and yeast transcription factor GCN4 (231-252).

28. The non-peptide analog of claim 24, wherein said TRAF6 binding domain is a TRAF6 binding domain from a protein selected from the group consisting of CD40, Receptor Activator of NF-κB, IL-1 receptor-associated kinase 1 (IRAK1), IL-1 receptor-associated kinase 2 (IRAK2), IRAK-M and RIP2.

29. The non-peptide analog of claim 24, wherein said TRAF6 binding domain comprises a sequence selected from the group consisting of SEQ ID NOs: 1-18.

30. The non-peptide analog of claim 24, wherein said polypeptide comprises a sequence selected from the group consisting of SEQ ID NOs: 19 and 20.

31. A method of inhibiting Receptor Activator of NF-κB Ligand (RANKL)-induced osteoclast differentiation, comprising the step of:
applying to cells the non-peptide analog of claim 24, wherein inhibition of interaction between Receptor Activator of NF-κB and TRAF6 by said non-peptide analog results in inhibition of RANKL-induced osteoclast differentiation.

32. The method of claim 31, wherein said osteoclast differentiation is induced by breast cancer cells.

33. A method of inhibiting osteoclast differentiation in an individual in need of such treatment, comprising the step of:
applying to said individual the non-peptide analog of claim 24, wherein inhibition of interaction between Receptor Activator of NF-κB and TRAF6 by said non-peptide analog results in inhibition of osteoclast differentiation.

34. The method of claim 33, wherein said individual has a disease selected from the group consisting of metabolic bone disorders, leukemia, multiple myeloma, arthritis, and metastatic cancer of the bone.

35. A pharmaceutical composition comprising the non-peptide analog of claim 24 and a pharmaceutically acceptable carrier.
